(19) United States
(12) Patent Application Publication
(10) Pub. No.: US 2014/0171713 A1
(43) Pub. Date: Jun. 19, 2014

(54) NANO SAPO-35 AND METHOD OF MAKING

(71) Applicant: UOP LLC, Des Plaines, IL (US)

(72) Inventors: Jaime G. Moscoso, Mt. Prospect, IL (US); Deng-Yang Jan, Elk Grove Village, IL (US); Nicholas J. Schoenfeldt, Chicago, IL (US)

(73) Assignee: UOP LLC, Des Plaines, IL (US)

(21) Appl. No.: 13/715,595

(22) Filed: Dec. 14, 2012

(51) Int. Cl. C01B 37/08; C07C 1/22

(52) U.S. Cl. CPC C01B 37/08; C07C 1/22
USPC 585/640; 423/700; 423/718; 423/703

(57) ABSTRACT

A porous crystalline nano metallo-alumino-phosphate molecular sieve is described. The molecular sieve has a framework composition on an anhydrous and calcined basis expressed by an empirical formula

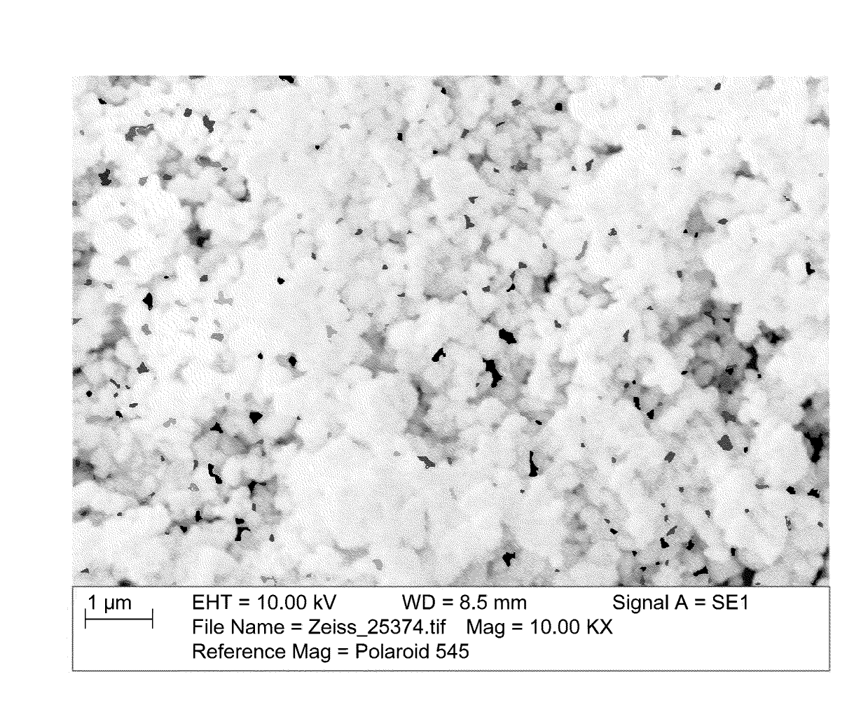

(ElₓAl_{y}P_{z})O₂

wherein El is silicon, magnesium, zinc, iron, cobalt, nickel, manganese, chromium, or combinations thereof, where x is the mole fraction of El and has a value from 0.001 to about 0.5, y is the mole fraction of Al and has a value of at least 0.01, z is the mole fraction of P has a value of at least 0.01, and x+y+z=1, where the molecular sieve is characterized as having a LEV framework and nano octahedral crystals with an average crystal size of less than 700 nm. Methods of making the molecular sieves, and methods of using the molecular sieves are also described.

### FIELD OF THE INVENTION

This invention relates generally to molecular sieves, and more particularly to a novel nano molecular sieve and a catalyst incorporating it, a process for producing the novel molecular sieve, and the use of the novel molecular sieve for converting oxygenates to light olefins.

### BACKGROUND OF THE INVENTION

Olefins are traditionally produced from petroleum feedstock by catalytic or steam cracking processes. These cracking processes, especially steam cracking, produce light olefin(s) such as ethylene and/or propylene from a variety of hydrocarbon feedstocks.

The limited supply and increasing cost of crude oil has prompted the search for alternative processes for producing hydrocarbon products. An important alternate feed for the production of light olefins is oxygenates, such as alcohols, particularly methanol and ethanol, ethers such as dimethyl ether, methyl ethyl ether, and diethyl ether, dimethyl carbonate, and methyl formate. These oxygenates may be produced by fermentation, or from synthesis gas derived from natural gas, petroleum liquids, carbonaceous materials, including coal, recycled plastics, municipal wastes, or other organic materials.

Oxygenates are converted to olefin products through a catalytic process. The conversion of a feed containing oxygenates is usually conducted in the presence of a molecular sieve catalyst. One process that is particularly useful in producing olefins is the conversion of methanol to hydrocarbons and especially to light olefins. The commercial interest in the methanol to olefins (MTO) process is based on the fact that methanol can be obtained from readily available raw materials, such as coal or natural gas, which are treated to produce synthesis gas, which is in turn processed to produce methanol.

Although ZSM-type molecular sieves and other molecular sieves may be used for the production of olefins from oxygenates, silicoaluminophosphate (SAPO) molecular sieves have been found to be of particular value in this catalytic process.

SAPOs are molecular sieves which have a three-dimensional microporous framework structure of AlO₂, PO₂, and SiO₂ tetrahedral oxide units.

Microporous silico-aluminophosphate (SAPO) molecular sieves are built of alumina, phosphate and silicate tetrahedral building units. They are manufactured from sources of silicon, such as a silica sol, aluminum, such as hydrated aluminum oxide, and phosphorus, such as orthophosphoric acid. The use of organic templates, such as tetraethylammonium hydroxide, isopropylamine or di-n-propylamine, plays a major role in synthesizing new molecular sieves.

SAPO-35 is a small-pore molecular sieve with two intersecting channels and eight member ring pore openings. SAPO-35 has two morphologies: spherical plates or cubic rhombohedra. The cubic morphology has a crystalline size of approximately 5-20 μm. Suitable organic templates for the synthesis of standard SAPO-35 include organic amines such as hexamethyleneimine, quinuclidine, cyclohexylamine, 2-methylcyclohexylamine, methylpiperidine, and piperidine. Crystallization of SAPO-35 is performed at 150-200° C., and it has a long crystallization period, typically 48 hr or more.

U.S. Pat. No. 4,440,871 describes one method of making SAPO-35. A reaction mixture of orthophosphoric acid, aluminum isopropoxide, silica, and quinuclidine is mixed and heated at 150-200° C. for 48-187 hrs.

Another method is described in U.S. Pat. No. 7,037,874; Venkatathri, synthesis, characterization, and catalytic properties of a LEV type silicoaluminophosphate molecular sieve, SAPO-35 from aqueous media using aluminum iospropoxide and hexamethyleneimine template, Applied Catalysis A: General 340 (2008) 265-270; and synthesis and characterization of high silica content silicoaluminophosphate SAPO-35 from non-aqueous medium, Catalysis Communications 7 (2006) 773-777, which are herein fully incorporated by reference. The process uses orthophosphoric acid, aluminum isopropoxide, and hexamethyleneimine. Crystallization takes 96 hrs or more, and the crystals are 12 μm.

However, additional methods of making ELAPO-35 and SAPO-35 are needed.

### SUMMARY OF THE INVENTION

One aspect of the invention is a porous crystalline metallo-alumino-phosphate molecular sieve. In one embodiment, the molecular sieve has a framework composition on an anhydrous and calcined basis expressed by an empirical formula

(ElₓAl_{y}P_{z})O₂

wherein El is selected from the group consisting of silicon, magnesium, zinc, iron, cobalt, nickel, manganese, chromium, or combinations thereof, where x is the mole fraction of El and has a value from 0.001 to about 0.5, y is the mole fraction of Al and has a value of at least 0.01, z is the mole fraction of P has a value of at least 0.01, and x+y+z=1, where the molecular sieve is characterized as having a LEV framework and octahedral crystals with an average crystal size of less than 700 nm.

Another aspect of the invention involves a process for the preparation of a porous crystalline metallo-alumino-phosphate molecular sieve having a framework composition on an anhydrous and calcined basis expressed by an empirical formula

(ElₓAl_{y}P_{z})O₂

wherein El is selected from the group consisting of silicon, magnesium, zinc, iron, cobalt, nickel, manganese, chromium, or combinations thereof, where x is the mole fraction of El and has a value of 0.001 to about 0.5, y is the mole fraction of Al and has a value of at least 0.01, z is the mole fraction of P has a value of at least of 0.01, and x+y+z=1. In one embodiment, the process includes providing a reaction mixture comprising an aluminum source, an El source, phosphorus source, a dual organic template source comprising a quaternary ammonium organic template source, and an organic amine template source; crystallizing the molecular sieves at a temperature between 100° C. to 200° C. to provide the molecular sieve; and calcining the molecular sieve in air, where the molecular sieve is characterized as having a LEV framework and nano octahedral crystals with an average crystal size of less than 700 nm.

Another aspect of the invention involves a process for converting oxygenates to light olefins. In one embodiment, the method includes contacting the oxygenates with a catalyst at conversion conditions, the catalyst comprising a crystalline metallo-alumino-phosphate molecular sieve having a framework composition on an anhydrous and calcined basis expressed by an empirical formula

(ElₓAl_{y}P_{z})O₂

wherein El is selected from the group consisting of silicon, magnesium, zinc, iron, cobalt, nickel, manganese, chromium, or combinations thereof, where x is the mole fraction of El and has a value from 0.001 to about 0.5, y is the mole fraction of Al and has a value of at least 0.01, z is the mole fraction of P has a value of at least 0.01, and x+y+z=1, where the molecular sieve is characterized as having a LEV framework and octahedral crystals with an average crystal size of less than 700 nm.

### BRIEF DESCRIPTION OF THE DRAWING

The FIGURE is an SEM of one embodiment of the present invention.

### DETAILED DESCRIPTION OF THE INVENTION

A new nano ElAPO-35 has been synthesized. Nano ElAPO-35 has nano octahedral crystals and a crystallite size less than about 700 nm. The use of dual templates makes the synthesis of nano ElAPO-35 possible. Nano ElAPO-35 uses a dual organic template source of quaternary ammonium organic template sources and organic amine template sources. Nano ElAPO-35 can be crystallized at temperatures less than about 200° C., and it has a crystallization period of less than about 24 hrs.

With favorable morphology and cost-effective synthesis conditions, nano ElAPO-35 presents an interesting material for hydrocarbon, carbonhydrate, and oxygenate conversions such as methanol conversion to olefins (MTO).

One aspect of the invention is a porous crystalline metallo-alumino-phosphate molecular sieve. The molecular sieve has a framework composition on an anhydrous and calcined basis expressed by an empirical formula

(ElₓAl_{y}P_{z})O₂

wherein El is selected from the group consisting of silicon, magnesium, zinc, iron, cobalt, nickel, manganese, chromium, or combinations thereof, where x is the mole fraction of El and has a value from 0.001 to about 0.5, y is the mole fraction of Al and has a value of at least 0.01, z is the mole fraction of P has a value of at least 0.01, and x+y+z=1, where the molecular sieve is characterized as having a LEV framework and nano octahedral crystals with an average crystal size of less than 700 nm.

In some embodiments, the molecular sieve is characterized in that it has the x-ray diffraction pattern having at least the d-spacings and intensities given in Table A below:

TABLE A

| 2Θ | d(Å) | I/Io |
|---|---|---|
| 8.66-8.71 | 10.13-10.2 | m |
| 10.87-10.93 | 8.08-8.12 | s |
| 11.38-11.42 | 7.7-7.76 | w |
| 13.51-13.57 | 6.51-6.54 | m |
| 16.07-16.13 | 5.48-5.5 | w |
| 17.12-17.16 | 5.16-5.17 | w-m |
| 17.37-17.43 | 5.08-5.09 | s-vs |
| 20.58-20.64 | 4.29-4.31 | m |
| 21.06-21.12 | 4.2-4.21 | w-m |
| 22.07-22.14 | 4.01-4.02 | vs |
| 23.52-23.58 | 3.76-3.77 | m |
| 24.68-24.75 | 3.59-3.6 | w |
| 27.2-27.28 | 3.26-3.27 | w |
| 27.9-27.95 | 3.18-3.19 | m |
| 28.25-28.32 | 3.14-3.15 | m |
| 32.44-32.52 | 2.75-2.75 | m |
| 34.04-34.12 | 2.62-2.63 | w |
| 41.79-41.85 | 2.15-2.16 | w |
| 42.22-42.3 | 2.13-2.14 | w |

The molecular sieve generally has an average crystal size less than 500 nm, or less than 300 nm, or less than 200 nm, or less than 100 nm.

Another aspect of the invention concerns the preparation of the nano ElAPO-35 molecular sieves described above. The process comprises providing a reaction mixture having an aluminum source, a phosphorus source, an El source, water, and a dual organic template source.

El is one or more elements chosen from silicon, magnesium, zinc, iron, cobalt, nickel, manganese, and chromium. Sources for elements “El” include oxides, hydroxides, alkoxides, nitrates, sulfates, halides, carboxylates, and mixtures thereof. When El is a mixture of metals, “x” represents the total amount of the metal mixture present. Preferred metals (El) are silicon, magnesium and cobalt, with silicon being especially preferred.

Suitable silicon sources include, but are not limited to, fumed, colloidal, or precipitated silica.

Preferred reactive sources of aluminum and phosphorus are pseudo-boehmite alumina and phosphoric acid, but organic phosphates or crystalline or amorphous aluminophosphates have been found satisfactory.

The dual organic template source comprises quaternary ammonium organic template sources and organic amine template sources. The ratio of quaternary ammonium organic template sources to organic amine template sources is typically in the range of about 2 to about 5, or about 2.5 to about 4.5, or about 3.0 to about 4.0, or about 3.3. There can be one or more quaternary ammonium organic template sources and one or more organic amine template sources.

Suitable quaternary ammonium organic template sources include, but are not limited to propyl trimethylammonium hydroxide, propyl trimethylammonium fluoride, propyl trimethylammonium bromide, propyl trimethylammonium chloride, propyl trimethylphosphonium hydroxide, diethyldimethylammonium hydroxide, dimethyldipropylammonium hydroxide, or combinations thereof.

Suitable organic amine template sources include, but are not limited to, dimethyldicyclohexylamine, tripropylamine, triethylamine, dipropylamine, propylamine, dimethylamine, diethylamine, or combinations thereof.

The dual organic template source (total amount of templating agent including both quaternary ammonium organic template sources and organic amine template sources) is supplied to the reaction mixture in a ratio from about 0.5 to about 1.5 times the amount of aluminum source on a molar basis, or about 1.0 to about 1.5, or about 1.2 to about 1.4, or about 1.3. The dual organic template source and phosphorus source are supplied to the mixture at a ratio, on a molar basis, of about 0.5 to about 1.5 times the amount of phosphorus source, or about 1.0 to about 1.5, or about 1.2 to about 1.4, or about 1.3.

The reaction mixture, including a source of aluminum, a source of phosphorus, the dual organic template source, and a source of one or more metals is placed in a sealed pressure vessel which is lined with an inert plastic material such as polytetrafluoroethylene, and heated preferably under autogenous pressure at a temperature of less than about 200° C., or between about 100° C. and less than about 200° C., or about 125° C. to about 175° C. for a time sufficient to produce crystals. Typically, the time is less than about 24 hr, or about 1 to less than about 24 hours, or about 1 to about 20 hours. The desired product is recovered by any convenient separation method such as centrifugation, filtration or decanting.

The molecular sieves of the present invention may be combined with one or more formulating agents to form a molecular sieve catalyst composition or a formulated molecular sieve catalyst composition. The formulating agents may be one or more of binding agents, matrix or filler materials, catalytically active materials, and mixtures thereof. This formulated molecular sieve catalyst composition is formed into desired shapes and sized particles by well-known techniques such as spray drying, pelletizing, extrusion, and the like.

Matrix materials are typically effective in: reducing overall catalyst cost; acting as thermal sinks assisting in shielding heat from the catalyst composition, for example during regeneration; densifying the catalyst composition; increasing catalyst strength, such as crush strength and attrition resistance; and controlling the rate of conversion in a particular process. Matrix materials include synthetic and naturally occurring materials such as clays, silica, and metal oxides. Clays include, but are not limited to, kaolin, kaolinite, montmorillonite, saponite, and bentonite.

Binders include any inorganic oxide well known in the art, and examples include, but are not limited to, alumina, silica, aluminum-phosphate, silica-alumina, and mixtures thereof. When a binder is used, the amount of molecular sieve present is in an amount from about 10 to 90 weight percent of the catalyst. Preferably, the amount of molecular sieve present is in an amount from about 30 to 70 weight percent of the catalyst.

The molecular sieve and the formulating agents are combined in a liquid to form slurry, and mixed to produce a substantially homogeneous mixture containing the molecular sieve. Examples of suitable liquids include water, alcohol, ketones, aldehydes, esters, and combinations thereof. The liquid is typically water.

The molecular sieve and the formulating agents may be in the same or different liquid, and may be combined in any order, together, simultaneously, sequentially, or a combination thereof. In some embodiments, the same liquid is used. The molecular sieve and formulating agents can be combined in a liquid as solids, substantially dry or in a dried form, or as slurries, together or separately. If solids are added together as dry or substantially dried solids, a limited and/or controlled amount of liquid can be added.

In some embodiments, the slurry of the molecular sieve and formulating agents is mixed or milled to achieve sufficiently uniform slurry of smaller particles that is then fed to a forming unit to produce the molecular sieve catalyst composition. A spray dryer is often used as the forming unit. Typically, the forming unit is maintained at a temperature sufficient to remove most of the liquid from the slurry and from the resulting molecular sieve catalyst composition. The resulting catalyst composition when formed in this way takes the form of microspheres.

Generally, the particle size of the powder is controlled to some extent by the solids content of the slurry. However, the particle size of the catalyst composition and its spherical characteristics are also controllable by varying the slurry feed properties and conditions of atomization. Also, although spray dryers produce a broad distribution of particle sizes, classifiers are normally used to separate the fines which can then be milled to a fine powder and recycled to the spray dryer feed mixture.

After the molecular sieve catalyst composition is formed in a substantially dry or dried state, a heat treatment, such as calcination, at an elevated temperature is usually performed to further harden and/or activate the formed catalyst composition. A conventional calcination environment is air that typically includes a small amount of water vapor. Typical calcination temperatures are in the range from about 400° C. to about 1000° C., or about 500° C. to about 800° C., or about 550° C. to about 700° C. The calcination environment is a gas such as air, nitrogen, helium, flue gas (combustion product lean in oxygen), or any combination thereof. Heating is carried out for a period of time typically from about 30 minutes to about 15 hours, or about 1 hour to about 10 hours, or about 1 hour to about 5 hours, or about 2 hours to about 4 hours.

In some embodiments, calcination of the formulated molecular sieve catalyst composition is carried out in any number of well known devices including rotary calciners, fluid bed calciners, batch ovens, and the like. Calcination time is typically dependent on the desired degree of hardening of the molecular sieve catalyst composition and the temperature.

In one embodiment, the molecular sieve catalyst composition is heated in air at a temperature of from about 600° C. to about 700° C. for about 2 to about 4 hr.

In addition to the molecular sieve of the present invention, the catalyst compositions of the present invention may comprise one or several other catalytically active materials.

The catalyst prepared in accordance with the present invention is useful in a process directed to the conversion of a feedstock comprising one or more oxygenates to one or more olefin(s). Preferably, the oxygenate in the feedstock comprises one or more alcohol(s), preferably aliphatic alcohol(s) where the aliphatic moiety of the alcohol(s) has from 1 to 20 carbon atoms, preferably from 1 to 10 carbon atoms, and most preferably from 1 to 4 carbon atoms. The alcohols useful as feedstock in the process of the invention include lower straight and branched chain aliphatic alcohols and their unsaturated counterparts.

Non-limiting examples of oxygenates include methanol, ethanol, n-propanol, isopropanol, methyl ethyl ether, dimethyl ether, diethyl ether, di-isopropyl ether, formaldehyde, dimethyl carbonate, dimethyl ketone, acetic acid, and mixtures thereof.

The feedstock, preferably comprising one or more oxygenates, is converted in the presence of a molecular sieve catalyst composition into one or more olefin(s) having 2 to 6 carbons atoms, preferably 2 to 5 carbon atoms. Most preferably, the olefin(s), alone or combination, are converted from a feedstock containing an oxygenate, preferably an alcohol, most preferably methanol, to the preferred olefin(s), ethylene and/or propylene.

The amount of light olefin(s) produced based on the total weight of hydrocarbon produced is at least 50 wt-%, preferably greater than 60 wt-%, more preferably greater than 70 wt-%. Higher yields may be obtained through improvements in the operation of the process as known in the art.

The feedstock may contain at least one or more diluents, typically used to reduce the concentration of the feedstock that is reactive toward the molecular sieve catalyst composition. Examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The preferred diluents are water and nitrogen, with water being particularly preferred. Water, can be used either in a liquid or a vapor form, or a combination thereof. The diluent can be added directly to a feedstock entering into a reactor, added directly into a reactor, or added with a molecular sieve catalyst composition. The amount of diluent in the feedstock is generally in the range of from about 5 to about 50 mol-% based on the total number of moles of the feedstock and diluent, and preferably from about 5 to about 35 mol-%.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like.

In one embodiment, a fluidized bed process or high velocity fluidized bed process includes a reactor system, a regeneration system and a product recovery system.

The fluidized bed reactor system has a first reaction zone within one or more riser reactor(s) and a second reaction zone within at least one disengaging vessel, preferably comprising one or more cyclones. The riser reactor(s) and disengaging vessel are contained within a single reactor vessel. Fresh feedstock is fed to the one or more riser reactor(s) in which a molecular sieve catalyst composition or coked version thereof is introduced. In one embodiment, the molecular sieve catalyst composition or coked version thereof is contacted with a liquid or gas, or combination thereof, prior to being introduced to the riser reactor(s), preferably the liquid is water or methanol, and the gas is an inert gas such as nitrogen. If regeneration is required, the aluminophosphate molecular sieve catalyst can be continuously introduced as a moving bed to a regeneration zone where it can be regenerated, such as, for example, by removing carbonaceous materials by oxidation in an oxygen-containing atmosphere. In some embodiments, the catalyst will be subject to a regeneration step by burning off carbonaceous deposits accumulated during reactions.

In converting methanol to olefins using the catalyst compositions of the invention, the process is preferably carried out in the vapor phase such that the feedstock is contacted in a vapor phase in a reaction zone with an aluminophosphate molecular sieve at effective process conditions such as to produce light olefins, i.e., an effective temperature, pressure, WHSV (weight hourly space velocity) and, optionally, an effective amount of diluent, correlated to produce light olefins. Alternatively, the process may be carried out in a liquid phase. When the process is carried out in the liquid phase, the process involves the separation of products formed in a liquid reaction media and can result in different conversions and selectivities of feedstock to product with respect to the relative ratios of the light olefin products as compared to that formed by the vapor phase process.

The temperatures which may be employed in the process may vary over a wide range depending, at least in part, on the selected aluminophosphate catalyst. In general, the process can be conducted at an effective temperature between about 200° C. and about 700° C., or about 250° C. and about 600° C., or about 300° C. and about 500° C. Temperatures outside these ranges are not excluded from the scope of this invention, although they do not fall within certain desirable embodiments of the invention. At the lower end of the temperature ranges and, thus, generally at the lower rate of reaction, the formation of the desired light olefin products may become markedly slow. At the upper end of the temperature range and beyond, the process may not form an optimum amount of light olefin products. Notwithstanding these factors, the reaction will still occur, and the feedstock can be converted to the desired light olefin products, at least in part, at temperatures outside the range between about 200° C. and about 700° C.

The process is effectively carried out over a wide range of pressures including autogenous pressures. At pressures between about 0.10 kPa (0.001 atmospheres) and about 101.3 mPa (1000 atmospheres), light olefin products will not necessarily form at all pressures. The preferred pressure is between about 1.01 kPa (0.01 atmospheres) and about 10.1 mPa (100 atmospheres). The pressures referred to herein for the process are exclusive of the inert diluent, if any is present, and refer to the partial pressure of the feedstock as it relates to methanol. Pressures outside the stated range are not excluded from the scope of this invention, although such do not fall within certain desirable embodiments of the invention. At the lower and upper end of the pressure range, light olefin products can be formed, but the process will not be optimum.

The process is run for a period of time sufficient to produce the desired light olefin products. In general, the residence time employed to produce the desired product can vary from seconds to a number of hours. It will be readily appreciated by one skilled in the art that the residence time will be determined to a significant extent by the reaction temperature, the aluminophosphate molecular sieve selected, the weight hourly space velocity (WHSV), the phase (liquid or vapor) selected and, perhaps, selected reactor design characteristics.

The process is effectively carried out over a wide range of WHSV for the feedstock and is generally between about 0.01 and about 100 hr⁻¹ and preferably between about 0.1 and about 40 hr⁻¹. Values above 100 hr⁻¹ may be employed and are intended to be covered by the instant process, although such are not preferred.

In one embodiment, the process is carried out under process conditions comprising a temperature between about 300° C. and about 500° C., a pressure between about 10.1 kPa (0.1 atmosphere) and about 10.1 mPa (100 atmospheres), utilizing a WHSV expressed in hr⁻¹ for each component of the feedstock having a value between about 0.1 and about 40. The temperature, pressure, and WHSV are each selected such that the effective process conditions, i.e., the effective temperature, pressure, and WHSV are employed in conjunction, i.e., correlated, with the selected silicoaluminophosphate molecular sieve and selected feedstock such that light olefin products are produced.

The structure of the nano SAPO-35 molecular sieve was determined by x-ray analysis. The x-ray patterns presented in the following examples were obtained using standard x-ray powder diffraction techniques. The radiation source was a high-intensity, x-ray tube operated at 45 kV and 35 ma. The diffraction pattern from the copper K-alpha radiation was obtained by appropriate computer based techniques. Flat compressed powder samples were continuously scanned at 2° to 56° (2θ). Interplanar spacings (d) in Angstrom units were obtained from the position of the diffraction peaks expressed as θ where θ is the Bragg angle as observed from digitized data. Intensities were determined by the integrated area of the diffraction peaks after subtracting background, “I₀” being the intensity of the strongest line or peak, and “I” being the intensity of each of the other peaks.

As will be understood by those of skill in the art, the determination of the parameter 2θ is subject to both human and mechanical error, which in combination can impose an uncertainty of about ±0.4° on each reported value of 2θ. This uncertainty is, of course, also manifested in the reported values of the d-spacings, which are calculated from the 2θ values. This imprecision is general throughout the art and is not sufficient to preclude the differentiation of the present crystalline materials from each other and from the compositions of the prior art. In some of the x-ray patterns reported, the relative intensities of the d-spacings are indicated by the notations vs, s, m, and w which represent very strong, strong, medium, and weak, respectively. In terms of 100×I/I₀, the above designations are defined as:

w=0-15

m=15-60

s=60-80

vs=80-100

### EXAMPLE

In a container, 15.85 g of orthophosphoric acid (85%) was combined with 37.56 g of propyltrimethylammonium hydroxide (20%) (Sachem Chemical). To this mixture, 6.23 g of colloidal silica (LUDOX® AS-40 available from Aldrich.) was added, followed by 9.98 g of alumina (Versal 251 available from UOP). Finally, 2.64 g of dimethylcyclohexylamine (DMCHA) and 7.64 g of water were added. The resulting gel was mixed for 30 minutes.

The gel was transferred to 3 parr reactors. The autoclaves were kept at 175° C. in a tumble oven for 16 hrs. The product was recovered by centrifugation and washed with water three times. The product was dried in an oven at 125° C. The product was identified as nano SAPO-35 by XRD, as shown in Table I. Elemental analysis of the dried powder showed 22.0% Al, 20.9% P and 4.38% Si. This corresponds to Al_{0.495}P_{0.409}Si_{0.094}O₂, expressed as normalized mole fraction. SEM of the nano SAPO-35 shows a material with nano octahedral crystals with dimensions less than 200 nm, as shown in the FIGURE. The nano SAPO-35 was calcined at 600° C. for 6 hrs. It had a BET surface area of 466 m²/g, and a micropore volume of 0.231 cc/g.

TABLE I

| 2Θ | d(Å) | I/Iₒ % |
|---|---|---|
| 8.71 | 10.13 | m |
| 10.93 | 8.08 | s |
| 11.42 | 7.74 | w |
| 13.57 | 6.51 | m |
| 16.13 | 5.48 | w |
| 17.16 | 5.16 | w |
| 17.43 | 5.08 | s |
| 20.64 | 4.29 | m |
| 21.12 | 4.20 | m |
| 22.14 | 4.01 | vs |
| 22.90 | 3.88 | w |
| 23.58 | 3.76 | m |
| 24.75 | 3.59 | w |
| 27.28 | 3.26 | w |
| 27.95 | 3.18 | m |
| 28.32 | 3.14 | m |
| 29.44 | 3.03 | w |
| 32.52 | 2.75 | m |
| 34.12 | 2.62 | w |
| 34.66 | 2.58 | w |

The nano SAPO-35 molecular sieve was used in a MTO process as described above. The nano-SAPO-35 resulted in increased light olefin (ethylene+propylene) selectivity at 99% conversion compared to a LEV structure-type zeolite framework. The increased light olefin selectivity was primarily the result of decreased light gas formation, which is typically also accompanied by reduced coking and deactivation rates.

### Selectivity (C wt %) at 99% Conversion

| | Ethylene + | | | |
|---|---|---|---|---|
| Catalyst | Propylene | C4 | C5+ | C1-C3 |
| Nano SAPO-35 | 41.2 | 21.6 | 26.3 | 8.4 |
| Levine zeolite | 26.3 | 25.4 | 25.4 | 19.2 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A porous crystalline metallo-alumino-phosphate molecular sieve having a framework composition on an anhydrous and calcined basis expressed by an empirical formula
(ElₓAl_{y}P_{z})O₂
wherein El is selected from the group consisting of silicon, magnesium, zinc, iron, cobalt, nickel, manganese, chromium, or combinations thereof, where x is the mole fraction of El and has a value from 0.001 to about 0.5, y is the mole fraction of Al and has a value of at least 0.01, z is the mole fraction of P has a value of at least 0.01, and x+y+z=1, where the molecular sieve is characterized as having a LEV framework and octahedral crystals with an average crystal size of less than 700 nm.

2. The molecular sieve of claim 1 characterized in that it has an x-ray diffraction pattern having at least d-spacings and intensities given in Table A below:
TABLE A

| 2Θ | d(Å) | I/Io |
|---|---|---|
| 8.66-8.71 | 10.13-10.2 | m |
| 10.87-10.93 | 8.08-8.12 | s |
| 11.38-11.42 | 7.7-7.76 | w |
| 13.51-13.57 | 6.51-6.54 | m |
| 16.07-16.13 | 5.48-5.5 | w |
| 17.12-17.16 | 5.16-5.17 | w-m |
| 17.37-17.43 | 5.08-5.09 | s-vs |
| 20.58-20.64 | 4.29-4.31 | m |
| 21.06-21.12 | 4.2-4.21 | w-m |
| 22.07-22.14 | 4.01-4.02 | vs |
| 23.52-23.58 | 3.76-3.77 | m |
| 24.68-24.75 | 3.59-3.6 | w |
| 27.2-27.28 | 3.26-3.27 | w |
| 27.9-27.95 | 3.18-3.19 | m |
| 28.25-28.32 | 3.14-3.15 | m |
| 32.44-32.52 | 2.75-2.75 | m |
| 34.04-34.12 | 2.62-2.63 | w |
| 41.79-41.85 | 2.15-2.16 | w |
| 42.22-42.3 | 2.13-2.14 | w |

3. The molecular sieve of claim 1 where the average crystal size is less than about 500 nm.

4. A process for the preparation of a porous crystalline metallo-alumino-phosphate molecular sieve having a framework composition on an anhydrous and calcined basis expressed by an empirical formula
(ElₓAl_{y}P_{z})O₂
wherein El is selected from the group consisting of silicon, magnesium, zinc, iron, cobalt, nickel, manganese, chromium, or combinations thereof, where x is the mole fraction of El and has a value of 0.001 to about 0.5, y is the mole fraction of Al and has a value of at least 0.01, z is the mole fraction of P has a value of at least of 0.01, and x+y+z=1, the process comprising:
providing a reaction mixture comprising an aluminum source, an El source, phosphorus source, a dual organic template source comprising a quaternary ammonium organic template source, and an organic amine template source; crystallizing the molecular sieves at a temperature between 100° C. to 200° C. to provide the molecular sieve; and calcining the molecular sieve in air, where the molecular sieve is characterized as having a LEV framework and octahedral crystals with an average crystal size of less than 700 nm.

5. The process of claim 4 wherein the molecular sieve is characterized in that it has an x-ray diffraction pattern having at least d-spacings and intensities given in Table A below
TABLE A

| 2Θ | d(Å) | I/Io |
|---|---|---|
| 8.66-8.71 | 10.13-10.2 | m |
| 10.87-10.93 | 8.08-8.12 | s |
| 11.38-11.42 | 7.7-7.76 | w |
| 13.51-13.57 | 6.51-6.54 | m |
| 16.07-16.13 | 5.48-5.5 | w |
| 17.12-17.16 | 5.16-5.17 | w-m |
| 17.37-17.43 | 5.08-5.09 | s-vs |
| 20.58-20.64 | 4.29-4.31 | m |
| 21.06-21.12 | 4.2-4.21 | w-m |
| 22.07-22.14 | 4.01-4.02 | vs |
| 23.52-23.58 | 3.76-3.77 | m |
| 24.68-24.75 | 3.59-3.6 | w |
| 27.2-27.28 | 3.26-3.27 | w |
| 27.9-27.95 | 3.18-3.19 | m |
| 28.25-28.32 | 3.14-3.15 | m |
| 32.44-32.52 | 2.75-2.75 | m |
| 34.04-34.12 | 2.62-2.63 | w |
| 41.79-41.85 | 2.15-2.16 | w |
| 42.22-42.3 | 2.13-2.14 | w |

6. The process of claim 4 wherein a ratio of the quaternary ammonium organic template source to the organic amine template source is in a range of about 2 to about 5.

7. The process of claim 4 wherein the dual organic template source is present in the reaction mixture in an amount on a molar basis from about 0.5 to about 1.5 times an amount of the aluminum source.

8. The process of claim 4 wherein the dual organic template source is present in the reaction mixture in an amount on a molar basis from about 0.5 to about 1.5 times an amount of the phosphorus source.

9. The process of claim 4 wherein the quaternary ammonium organic template source is selected from the group consisting of propyl trimethylammonium hydroxide, propyl trimethylammonium fluoride, propyl trimethylammonium bromide, propyl trimethylammonium chloride, propyl trimethylphosphonium hydroxide, diethydimethylammonium hydroxide, dimethyldipropylammonium hydroxide, or combinations thereof.

10. The process of claim 9 wherein the quaternary ammonium organic template source is propyl trimethylammonium hydroxide.

11. The process of claim 4 wherein the organic amine template source is selected from the group consisting of dimethylcyclohexylamine, tripropylamine, triethylamine, dipropylamine, propylamine, dimethylamine, diethylamine, or combinations thereof.

12. The process of claim 11 wherein the organic amine template source is dimethylcyclohexylamine.

13. The process of claim 4 wherein the reaction mixture is crystallized at the temperature in the range of about 125° C. to about 175° C. for a period of about 1 day or less.

14. The process of claim 4 where the average crystal size is less than about 500 nm.

15. The process of claim 4 wherein the molecular sieve is calcined at a temperature in a range of about 550° C. to about 650° C.

16. A process for converting oxygenates to light olefins comprising contacting the oxygenates with a catalyst at conversion conditions, the catalyst comprising a crystalline metallo-alumino-phosphate molecular sieve having a framework composition on an anhydrous and calcined basis expressed by an empirical formula
(ElₓAl_{y}P_{z})O₂
wherein El is selected from the group consisting of silicon, magnesium, zinc, iron, cobalt, nickel, manganese, chromium, or combinations thereof, where x is the mole fraction of El and has a value from 0.001 to about 0.5, y is the mole fraction of Al and has a value of at least 0.01, z is the mole fraction of P has a value of at least 0.01, and x+y+z=1, where the molecular sieve is characterized as having a LEV framework and octahedral crystals with an average crystal size of less than 700 nm.

17. The process of claim 16 wherein the molecular sieve is characterized in that it has an x-ray diffraction pattern having at least d-spacings and intensities given in Table A below:
TABLE A

| 2Θ | d(Å) | I/Io |
|---|---|---|
| 8.66-8.71 | 10.13-10.2 | m |
| 10.87-10.93 | 8.08-8.12 | s |
| 11.38-11.42 | 7.7-7.76 | w |
| 13.51-13.57 | 6.51-6.54 | m |
| 16.07-16.13 | 5.48-5.5 | w |
| 17.12-17.16 | 5.16-5.17 | w-m |
| 17.37-17.43 | 5.08-5.09 | s-vs |
| 20.58-20.64 | 4.29-4.31 | m |
| 21.06-21.12 | 4.2-4.21 | w-m |
| 22.07-22.14 | 4.01-4.02 | vs |
| 23.52-23.58 | 3.76-3.77 | m |
| 24.68-24.75 | 3.59-3.6 | w |
| 27.2-27.28 | 3.26-3.27 | w |
| 27.9-27.95 | 3.18-3.19 | m |
| 28.25-28.32 | 3.14-3.15 | m |
| 32.44-32.52 | 2.75-2.75 | m |
| 34.04-34.12 | 2.62-2.63 | w |
| 41.79-41.85 | 2.15-2.16 | w |
| 42.22-42.3 | 2.13-2.14 | w |

18. The process of claim 16 wherein the catalyst further comprises an inorganic oxide binder.

19. The process of claim 16 where the average crystal size is less than about 500 nm.

20. The process of claim 16 wherein the conversion conditions include a temperature in the range of about 200° C. to about 700° C., a pressure in the range of about 0.10 kPa to about 101.3 mPa, and a WHSV in a range of about 0.01 to about 100 hr⁻¹.
